SEQUENCE LISTING (17 sequences)

SEQ ID NO: 1 - protein, 775 aa, Thermococcus sp.
  source 1..775: note = strain 9oN-7
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 2 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 3 - protein, 777 aa, synthetic construct
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKKGS

SEQ ID NO: 4 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 5 - protein, 774 aa, Thermococcus kodakarensis
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 6 - protein, 773 aa, Pyrococcus abyssi
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVDIETLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPVIRRTINLPTYTLEAVYEAIFGKSKEKVYAHEIAEAWETGKGLERVAKYSMEDAKVTFELGKEFFPMEAQLARLVGQPVWDVSRSSTGNLVEWFLLRKAYERNELAPNKPDEREYERRLRESYEGGYVKEPEKGLWEGIVSLDFRSLYPSIIITHNVSPDTLNRENCKEYDVAPQVGHRFCKDFPGFIPSLLGNLLEERQKIKKRMKESKDPVEKKLLDYRQRAIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLKYQKTKQVGLGAWLKFGS

SEQ ID NO: 7 - protein, 775 aa, Thermococcus waiotapuensis
MILDADYITEDGKPVIRVFKKEKGEFKINYDRDFEPYIYALLKDDSAIEDIKKITAERHGTTVRVTRAERVKKKFLGRPVEVWKLYFTHPQDVPAIRDKIREHPAVVDIYEYDIPFAKRYLIDKGLIPMEGNEELRMLAFDIETLYHEGEEFGEGPILMISYADEEGARVITWKNIDLPYVESVSTEKEMIKRFLKVIQEKDPDVLITYNGDNFDFAYLKKRSETLGVKFILGRDGSEPKIQRMGDRFAVEVKGRIHFDLYPVIRRTINLPTYTLETVYEAIFGQPKEKVYAEEIAQAWESGEGLERVARYSMEDAKATYELGKEFFPMEAQLSRLVGQSLWDVSRSSTGNLVEWFLLRKAYERNELAPNKPDERELARRAESYAGGYVKEPEKGLWENIVYLDYKSLYPSIIITHNVSPDTLNREGCREYDVAPQVGHRFCKDFPGFIPSLLGDLLEERQKVKKKMKATVDPIERKLLDYRQRAIKILANSYYGYYGYANARWYCRECAESVTAWGRQYIETTMREIEEKFGFKVLYADTDGFFATIPGADAETVKKKTKEFLNYINPRLPGLLELEYEGFYRRGFFVTKKKYAVIDEEDKITTRGLEIVRRDWSEIAKETQARVLEAILKHGDVEEAVRIVKEVTEKLSRYEVPPEKLVIYEQITRNLRDYRATGPHVAVAKRLAARGIKIRPGTVISYIVLKGPGRVGDRAIPFDEFDPAKHRYDAEYYIENQVLPAVERILRAFGYRKEDLRYQKTKQAGLGAWLKPKTGS

SEQ ID NO: 8 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 9 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSSGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 10 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 11 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSNGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 12 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEGVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 13 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEFVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 14 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKTGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 15 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVSGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 16 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLCDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 17 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLFDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

(19) United States
(12) Patent Application Publication
(10) Pub. No.: US 2025/0059521 A1
(43) Pub. Date: Feb. 20, 2025

(54) POLYMERASES, COMPOSITIONS, AND METHODS OF USE

(71) Applicants: ILLUMINA, INC., San Diego, CA (US); ILLUMINA CAMBRIDGE LIMITED, Cambridge (GB); ILLUMINA SINGAPORE PTE. LTD., Singapore (SG)

(72) Inventors: Kay Klausing, San Diego, CA (US); Hamed Tabatabaei Ghomi, Cambridge (GB); Misha Golynskiy, San Diego, CA (US); Saurabh Nirantar, Singapore (SG); Seth McDonald, San Diego, CA (US); Sergio Peisajovich, San Diego, CA (US)

(73) Assignees: ILLUMINA, INC., San Diego, CA (US); ILLUMINA CAMBRIDGE LIMITED, Cambridge (GB); ILLUMINA SINGAPORE PTE. LTD., Singapore (SG)

(21) Appl. No.: 18/824,208

(22) Filed: Sep. 4, 2024

Related U.S. Application Data
(63) Continuation of application No. 18/120,639, filed on Mar. 13, 2023, now Pat. No. 12,104,182.
Application No. 18/120,639 is a continuation of application No. 17/226,393, filed on Apr. 9, 2021, now Pat. No. 11,634,697.
Application No. 17/226,393 is a continuation of application No. 16/703,569, filed on Dec. 4, 2019, now Pat. No. 11,001,816.
(60) Provisional application No. 62/775,662, filed on Dec. 5, 2018.

(51) Int. Cl. C12N 9/12

(52) U.S. Cl. CPC C12N 9/1252; C12Y 207/07007

(57) ABSTRACT

Presented herein are altered polymerase enzymes for improved incorporation of nucleotides and nucleotide analogues, in particular altered polymerases that maintain high fidelity under reduced incorporation times, as well as methods and kits using the same.

### PRIORITY

This application is a continuation of U.S. application Ser. No. 18/120,639, filed Mar. 13, 2023, which is a continuation of U.S. application Ser. No. 17/226,393, filed Apr. 9, 2021, now issued U.S. Pat. No. 11,634,697, which issued on Apr. 25, 2023, which is a continuation of U.S. application Ser. No. 16/703,569, filed Dec. 4, 2019, now issued U.S. Pat. No. 11,001,816, which issued on May 11, 2021, which claims the benefit of U.S. Provisional Application No. 62/775,662, filed Dec. 5, 2018, the disclosures of which are incorporated by reference herein in their entireties.

### SEQUENCE LISTING

This application contains a Sequence Listing electronically submitted to the United States Patent and Trademark Office via Patent Center as an XML file entitled “0531001727US04.xml” having a size of 679,234 bytes and created on Nov. 9, 2024. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### FIELD

The present disclosure relates to, among other things, altered polymerases for use in performing a nucleotide incorporation reaction, particularly in the context of nucleic acid sequencing by synthesis.

### BACKGROUND

Next-generation sequencing (NGS) technology relies on DNA polymerases as a critical component of the sequencing process. Reduction of the time for sequencing a template while maintaining high fidelity is desirable. Reducing each cycle of a sequencing by synthesis (SBS) process is a useful step to achieving a shorter sequencing run time. One approach to reduce cycle time is to reduce the time of the incorporation step. However, while reductions in incorporation time could offer significant improvement to the overall run time, they typically do so at the expense of fidelity. For instance, phasing rates, pre-phasing rates, and/or bypass rates increase, and as a consequence error rate is increased. At low error rates, during a sequencing run most template molecules in a cluster terminate in the same labeled nucleotide and the signal is clear. In contrast, at reduced fidelity, during a sequencing run an increasing number of template molecule in a cluster terminate in the incorrect labeled nucleotide and the signal can become too noisy to accurately determine which nucleotide was incorporated.

### SUMMARY

Provided herein are recombinant DNA polymerases. One example of a polymerase of the present disclosure includes an amino acid sequence that is at least 80% identical to a 9°N DNA polymerase amino acid sequence SEQ ID NO:1.

Another example of a polymerase of the present disclosure of a DNA polymerase includes an amino acid sequence that is at least 80% identical to a 9°N DNA polymerase amino acid sequence SEQ ID NO:1, and an amino acid substitution mutation at a position functionally equivalent to Ala281, Phe283, Thr349, or Trp397 in the 9°N DNA polymerase amino acid sequence.

In one embodiment, a DNA polymerase includes an amino acid sequence that is at least 80% identical to a 9°N DNA polymerase amino acid sequence SEQ ID NO:8, and an amino acid substitution mutation at a position functionally equivalent to Ala281Gly, Ala281Phe, Phe283Ser, Thr349Ser, Thr349Asn, Thr349Lys, Trp397Cys, Trp397Phe, or His633Thr in the 9°N DNA polymerase amino acid sequence, In one embodiment, a DNA polymerase includes the amino acid sequence of any one of SEQ ID NOs:9-17.

Also provided is a nucleic acid molecule encoding a polymerase described herein, an expression vector that includes the nucleic acid molecule, and a host cell that includes the vector.

Further provided are methods. In one embodiment, a method is for incorporating modified nucleotides into DNA, and includes allowing the following components to interact: (i) a polymerase described herein, (ii) a DNA template; and (iii) a nucleotide solution.

Also provided are kits. In one embodiment, a kit is for performing a nucleotide incorporation reaction. The kit can include a polymerase described herein and a nucleotide solution.

### BRIEF DESCRIPTION OF THE DRAWINGS

FIG. 1 is a schematic showing alignment of polymerase amino acid sequences from Thermococcus sp. 9°N-7 (9°N, SEQ ID NO:1), Thermococcus litoralis (Vent, SEQ ID NO:2 and Deep Vent, SEQ ID NO:3), Thermococcus waiotapuensis (Twa, SEQ ID NO:7), Thermococcus kodakarensis (KOD, SEQ ID NO:5), Pyrococcus furiosus (Pfu, SEQ ID NO:4), Pyrococcus abyssi (Pab, SEQ ID NO:6). An “*” (asterisk) indicates positions which have a single, fully conserved residue between all polymerases. A “:” (colon) indicates conservation between groups of strongly similar properties as below—roughly equivalent to scoring >0.5 in the Gonnet PAM 250 matrix. A “.” (period) indicates conservation between groups of weakly similar properties as below—roughly equivalent to scoring=<0.5 and >0 in the Gonnet PAM 250 matrix.

FIG. 2 shows the error rates and phasing levels of selected altered polymerases of the present disclosure. Starting at the bottom of the figure, “slow” refers to slow incorporation time, i.e., the standard incorporation time of 46 seconds; “fast” refers to the faster incorporation time of 16 seconds; “1671”, “1901”, etc., identify specific altered polymerases; WT, T349K, T349N, T349S, etc., refer to specific mutations in an altered polymerase relative to Pol 1671 (“WT” corresponds to Pol 1671 for the purposes of this application); and Pols 1671 and e812 refer to control polymerases.

FIG. 3 compares various error metrics of polymerases 1671 (two different lots) and 1901 at different incorporation speeds. Polymerase 812 was used as a control. In the first row, “46s”, “22s”, “16s”, “12s”, and “10s” refer to the total incorporation time. In the second row, “30-8-8”, “14-4-4”, etc. refer to the static incubation period (in seconds) followed by the two mixing periods (also in seconds). The third row refers to the identities of the altered polymerases. On the Y axis, “PhiX Err” refers to the observed error rates; “Phas” refers to the observed phasing values; “Pre-Phas” refers to the observed pre-phasing values; and “Q30” refers to the percentage of reads that pass the Q30 quality filter, i.e. an error rate of less of equal to 1 in 1000, or 0.1%.

### DETAILED DESCRIPTION OF EXEMPLARY EMBODIMENTS

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Conditions that are “suitable” for an event to occur or “suitable” conditions are conditions that do not prevent such events from occurring. Thus, these conditions permit, enhance, facilitate, and/or are conducive to the event.

As used herein, “providing” in the context of a composition, an article, a nucleic acid, or a nucleus means making the composition, article, nucleic acid, or nucleus, purchasing the composition, article, nucleic acid, or nucleus, or otherwise obtaining the compound, composition, article, or nucleus.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Maintaining or surpassing current levels of performance at faster incorporation times can be aided by a new generation of polymerases. Presented herein are polymerase enzymes having significantly improved performance under sequencing by synthesis (SBS) fast cycle time conditions. The inventors have surprisingly identified certain altered polymerases which exhibit improved characteristics including improved accuracy during short incorporations times. Improved accuracy includes reduced error rate and reduced phasing. The altered polymerases have a number of other associated advantages, including reduced prephasing, reduced bypass rate, and improved quality metrics in SBS reactions. This improvement is maintained even when a polymerase is used at lower concentrations. Accordingly, in one embodiment, the concentration of a DNA polymerase in an SBS reaction can be from 120 ng/μl to 80 ng/μl. In one embodiment, the concentration of a DNA polymerase in a SBS reaction can be no greater than 120 ng/μl, no greater than 110 ng/μl, no greater than 100 ng/μl, or no greater than 90 ng/μl. In one embodiment, the concentration of a DNA polymerase in an SBS reaction can be at least 80 ng/μl, at least 90 ng/μl, at least 100 ng/μl, or at least 110 ng/μl.

Error rate refers to a measurement of the frequency of error in the identification of the correct base, i.e., the complement of the template sequence at a specific position, during a sequencing reaction. The fidelity with which a sequenced library matches the original genome sequence can vary depending on the frequency of base mutation occurring at any stage from the extraction of the nucleic acid to its sequencing on a sequencing platform. This frequency places an upper limit on the probability of a sequenced base being correct. In some embodiments, the quality score is presented as a numerical value. For example, the quality score can be quoted as QXX where the XX is the score and it means that that particular call has a probability of error of 10^{-XX/10}. Thus, as an example, Q30 equates to an error rate of 1 in 1000, or 0.1%, and Q40 equates to an error rate of 1 in 10,000, or 0.01%.

Phasing and pre-phasing are terms known to those of skill in the art and are used to describe the loss of synchrony in the readout of the sequence copies of a cluster. Phasing and pre-phasing cause the extracted intensities for a specific cycle to include the signal of the current cycle and noise from the preceding and following cycles. Thus, as used herein, the term “phasing” refers to a phenomenon in SBS that is caused by incomplete incorporation of a nucleotide in some portion of DNA strands within clusters by polymerases at a given sequencing cycle, and is thus a measure of the rate at which single molecules within a cluster lose sync with each other. Phasing can be measured during detection of cluster signal at each cycle and can be reported as a percentage of detectable signal from a cluster that is out of synchrony with the signal in the cluster. As an example, a cluster is detected by a “green” fluorophore signal during cycle N. In the subsequent cycle (cycle N+1), 99.9% of the cluster signal is detected in the “red” channel and 0.1% of the signal remains from the previous cycle and is detected in the “green” channel. This result would indicate that phasing is occurring, and can be reported as a numerical value, such as a phasing value of 0.1, indicating that 0.1% of the molecules in the cluster are falling behind at each cycle.

The term “pre-phasing” as used herein refers to a phenomenon in SBS that is caused by the incorporation of nucleotides without effective 3′ terminators, causing the incorporation event to go one cycle ahead. As the number of cycles increases, the fraction of sequences per cluster affected by phasing increases, hampering the identification of the correct base. Pre-phasing can be detected by a sequencing instrument and reported as a numerical value, such as a pre-phasing value of 0.1, indicating that 0.1% of the molecules in the cluster are running ahead at each cycle.

Detection of phasing and pre-phasing can be performed and reported according to any suitable methodology as is known in the art, for example, as described in U.S. Pat. No. 8,965,076 and U.S. Provisional Patent No. 62/535,558. For example, as described in the Examples below, phasing is detected and reported routinely during SBS sequencing runs on sequencing instrument such as HiSeq™ Genome Analyzer™, NextSeq™, NovaSeq™, iSeq™, MiniSeq™, or MiSeq™ sequencing platforms from Illumina, Inc. (San Diego, CA) or any other suitable instrument known in the art.

Reduced cycle times can increase the occurrence of phasing, pre-phasing, and/or bypass rate, each of which contributes to error rate. The discovery of altered polymerases which decrease the incidence of phasing, pre-phasing, and/or bypass rate, even when used in fast cycle time conditions, is surprising and provides a great advantage in SBS applications. For example, the altered polymerases can provide faster SBS cycle time, lower phasing and pre-phasing values, and/or longer sequencing read length. The characterization of error rate and phasing for altered polymerases as provided herein is set forth in the Example section below.

### Polymerases

Provided herein are polymerases, compositions including a polymerase, and methods of using a polymerase. A polymerase described herein is a DNA polymerase. In one embodiment, a polymerase of the present disclosure, also referred to herein as an “altered polymerase,” is based on the amino acid sequence of a reference polymerase. An altered polymerase includes substitution mutations at one or more residues when compared to the reference polymerase. A substitution mutation can be at the same position or a functionally equivalent position compared to the reference polymerase. Reference polymerases and functionally equivalent positions are described in detail herein. The skilled person will readily appreciate that an altered polymerase described herein is not naturally occurring.

A reference polymerase described herein has error rates that are useful is SBS reactions; however, using a reference polymerase in SBS reactions with shorter incorporation times increases the error rate. An altered polymerase described herein maintains the superior error rates observed with reference polymerases even when the altered polymerase is used in SBS reactions with shorter incorporation times. In one embodiment, reduced error rates occur when the altered polymerase is tested using fast incorporation times. Incorporation refers to the amount of time a DNA polymerase is in contact with a template. As used herein, a slow incorporation time is the incorporation time used under a standard cycle using a MiniSeq™ benchtop sequencing system. Slow incorporation times include from 40 seconds to 50 seconds. As used herein, a fast cycle time refers to an incorporation step that is from 10 seconds to 40 seconds. In one embodiment, a fast cycle time is an incorporation time of no greater than 40 seconds, no greater than 30 seconds, no greater than 20 seconds, no greater than 18 seconds, no greater than 16 seconds, no greater than 14 seconds, or no greater than 12 seconds. In one embodiment, a fast cycle time is an incorporation time of at least 10 seconds, at least 12 seconds, at least 14 seconds, at least 16 seconds, at least 18 seconds, at least 20 seconds, or at least 30 seconds. In one embodiment, a fast cycle time is an incorporation time of less than 40 seconds, less than 30 seconds, less than 20 seconds, less than 18 seconds, less than 16 seconds, less than 14 seconds, less than 12 seconds, or less than 10 seconds.

An altered polymerase described herein can be used in SBS reactions for runs of different lengths. A “run” refers to the number of nucleotides that are identified on a template. A run typically includes a run based on the first primer (e.g., a read1 primer) which reads one strand of a template and a run based on the second primer (e.g., a read2 primer) which reads the complementary strand of the template. In one embodiment, the number of nucleotides identified using the first primer or the second primer can be from 10 to 150 nucleotides. In one embodiment, the number of nucleotides identified using the first primer or the second primer can be no greater than 150 nucleotides, no greater than 130 nucleotides, no greater than 110 nucleotides, no greater than 90 nucleotides, no greater than 70 nucleotides, no greater than 50 nucleotides, no greater than 30 nucleotides, or no greater than 20 nucleotides. In one embodiment, the number of nucleotides identified using the first primer or the second primer can be at least 10, at least 20, at least 30, at least 50, at least 70, at least 90, at least 110, or at least 130 nucleotides.

In certain embodiments, an altered polymerase is based on a family B type DNA polymerase. An altered polymerase can be based on, for example, a family B archaeal DNA polymerase, a human DNA polymerase-α, or a phage polymerase.

Family B archaeal DNA polymerases are well known in the art as exemplified by the disclosure of U.S. Pat. No. 8,283,149. In certain embodiments, an archaeal DNA polymerase is from a hyperthermophilic archaeon and is thermostable.

In certain embodiments, a family B archaeal DNA polymerase is from a genus such as, for example, Thermococcus, Pyrococcus, or Methanococcus. Members of the genus Thermococcus are well known in the art and include, but are not limited to T. 4557, T. barophilus, T. gammatolerans, T. onnurineus, T. sibiricus, T. kodakarensis, T. gorgonarius, and T. waiotapuensis. Members of the genus Pyrococcus are well known in the art and include, but are not limited to P. NA2, P. abyssi, P. furiosus, P. horikoshii, P. yayanosii, P. endeavori, P. glycovorans, and P. woesei. Members of the genus Methanococcus are well known in the art and include, but are not limited to M. aeolicus, M maripaludis, M. vannielii, M voltae, M. thermolithotrophicus, and M jannaschii.

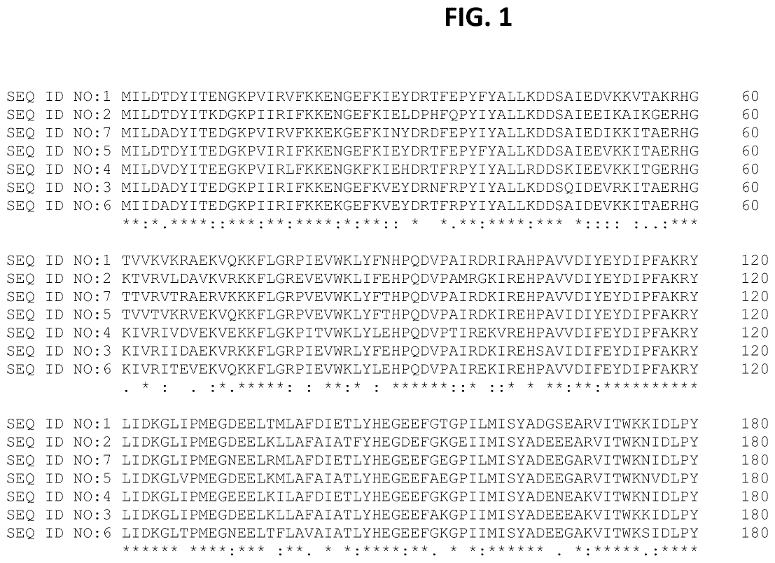

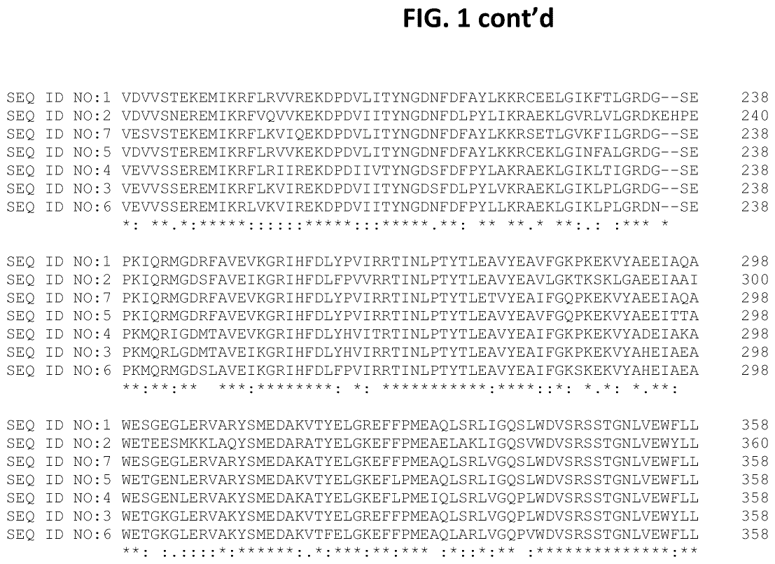

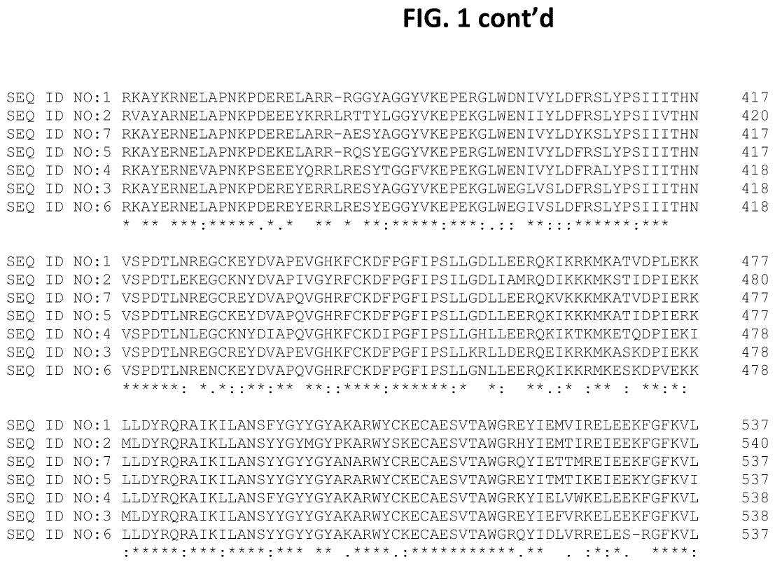

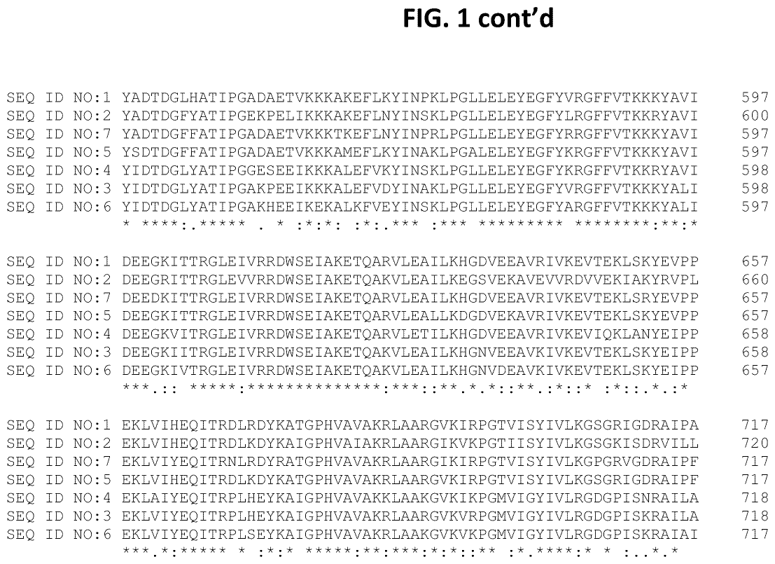

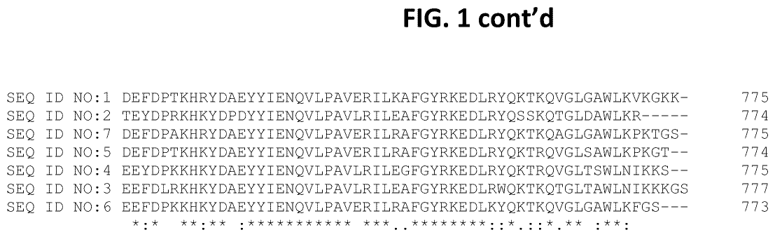

In one embodiment an altered polymerase is based on Vent®, Deep Vent®, 9°N, Pfu, KOD, or a Pab polymerase. Vent® and Deep Vent® are commercial names used for family B DNA polymerases isolated from the hyperthermophilic archaeon Thermococcus litoralis. 9°N polymerase is a family B polymerase isolated from Thermococcus sp. Pfu polymerase is a family B polymerase isolated from Pyrococcus furiosus. KOD polymerase is a family B polymerase isolated from Thermococcus kodakarensis. Pab polymerase is a family B polymerase isolated from Pyrococcus abyssi. Twa is a family B polymerase isolated from T. waiotapuensis. Examples of Vent®, Deep Vent®, 9°N, Pfu, KOD, Pab, and Twa polymerases are disclosed in FIG. 1.

In certain embodiments, a family B archaeal DNA polymerase is from a phage such as, for example, T4, RB69, or phi29 phage.

FIG. 1 shows a sequence alignment for proteins having the amino acid sequences shown in SEQ ID NOs:1-7. The alignment indicates amino acids that are conserved in the different family B polymerases. The skilled person will appreciate that the conserved amino acids and conserved regions are most likely conserved because they are important to the function of the polymerases, and therefore show a correlation between structure and function of the polymerases. The alignment also shows regions of variability across the different family B polymerases. A person of ordinary skill in the art can deduce from such data regions of a polymerase in which substitutions, particularly conservative substitutions, may be permitted without unduly affecting biological activity of the altered polymerase.

An altered polymerase described herein is based on the amino acid sequence of a known polymerase (also referred to herein as a reference polymerase) and further includes substitution mutations at one or more residues. In one embodiment, a substitution mutation is at a position functionally equivalent to an amino acid of a reference polymerase. By “functionally equivalent” it is meant that the altered polymerase has the amino acid substitution at the amino acid position in the reference polymerase that has the same functional role in both the reference polymerase and the altered polymerase.

In general, functionally equivalent substitution mutations in two or more different polymerases occur at homologous amino acid positions in the amino acid sequences of the polymerases. Hence, use herein of the term “functionally equivalent” also encompasses mutations that are “positionally equivalent” or “homologous” to a given mutation, regardless of whether or not the particular function of the mutated amino acid is known. It is possible to identify the locations of functionally equivalent and positionally equivalent amino acid residues in the amino acid sequences of two or more different polymerases on the basis of sequence alignment and/or molecular modelling. An example of sequence alignment to identify positionally equivalent and/or functionally equivalent residues is set forth in FIG. 1. For example, the residues in the Twa, KOD, Pab, Pfu, Deep Vent, and Vent polymerases of FIG. 1 that are vertically aligned are considered positionally equivalent as well as functionally equivalent to the corresponding residue in the 9°N polymerase amino acid sequence. Thus, for example residue 349 of the 9°N, Twa, KOD, Pfu, Deep Vent, and Pab polymerases and residue 351 of the Vent polymerase are functionally equivalent and positionally equivalent. Likewise, for example residue 633 of the 9°N, Twa, KOD, and Pab polymerases, residue 634 of the Pfu and Deep Vent polymerases, and residue 636 of the Vent polymerase are functionally equivalent and positionally equivalent. The skilled person can easily identify functionally equivalent residues in DNA polymerases.

In certain embodiments, the substitution mutation comprises a mutation to a residue having a non-polar side chain. Amino acids having non-polar side chains are well-known in the art and include, for example: alanine, glycine, isoleucine, leucine, methionine, phenylalanine, proline, tryptophan, and valine.

In certain embodiments, the substitution mutation comprises a mutation to a residue having a polar side chain. Amino acids having polar side chains are well-known in the art and include, for example: arginine, asparagine, aspartic acid, glutamine, glutamic acid, histidine, lysine, serine, cysteine, tyrosine, and threonine.

In certain embodiments, the substitution mutation comprises a mutation to a residue having a hydrophobic side chain. Amino acids having hydrophobic side chains are well-known in the art and include, for example: glycine, alanine, valine, leucine, isoleucine, proline, phenylalanine, methionine, and tryptophan.

In certain embodiments, the substitution mutation comprises a mutation to a residue having an uncharged side chain. Amino acids having uncharged side chains are well-known in the art and include, for example: glycine, serine, cysteine, asparagine, glutamine, tyrosine, and threonine, among others.

In one embodiment, an altered polymerase has an amino acid sequence that is structurally similar to a reference polymerase disclosed herein. In one embodiment, a reference polymerase is one that includes the amino acid sequence of 9°N (SEQ ID NO:1). In one embodiment, the reference polymerase is SEQ ID NO:1 with the following substitution mutations: Met129Ala, Asp141Ala, Glu43Ala, Cys223Ser, Leu408Ala, Tyr409Ala, Pro410Ile, Ala485Val, Tyr497Gly, Arg247Tyr, Glu599Asp, and His633Gly. This second reference polymerase is disclosed at SEQ ID NO:8, and is also referred to herein as the Pol 1671 polymerase. Other reference sequences include SEQ ID NO:2, 3, 4, 5, 6, or 7. Optionally, a reference polymerase is SEQ ID NO: 2, 3, 4, 5, 6, or 7 with substitution mutations functionally equivalent to the following substitution mutations in SEQ ID NO:1: Met129Ala, Asp141Ala, Glu143Ala, Cys223Ser, Leu408Ala, Tyr409Ala, Pro410Ile, Ala485Val, Tyr497Gly, Arg247Tyr, Glu599Asp, and His633Gly.

As used herein, an altered polymerase may be “structurally similar” to a reference polymerase if the amino acid sequence of the altered polymerase possesses a specified amount of sequence similarity and/or sequence identity compared to the reference polymerase.

Structural similarity of two amino acid sequences can be determined by aligning the residues of the two sequences (for example, a candidate polymerase and a reference polymerase described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polymerase is the polymerase being compared to the reference polymerase. A candidate polymerase that has structural similarity with a reference polymerase and polymerase activity is an altered polymerase.

Unless modified as otherwise described herein, a pair-wise comparison analysis of amino acid sequences or nucleotide sequences can be conducted, for instance, by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Current Protocols in Molecular Biology, Ausubel et al., eds., Current Protocols, a joint venture between Greene Publishing Associates, Inc. and John Wiley & Sons, Inc., supplemented through 2004).

One example of an algorithm that is suitable for determining structural similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., J. Mol. Biol. 215:403-410 (1990)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a protein may be selected from other members of the class to which the amino acid belongs. For example, it is well-known in the art of protein biochemistry that an amino acid belonging to a grouping of amino acids having a particular size or characteristic (such as charge, hydrophobicity, or hydrophilicity) can be substituted for another amino acid without altering the activity of a protein, particularly in regions of the protein that are not directly associated with biological activity. For example, non-polar amino acids include alanine, glycine, isoleucine, leucine, methionine, phenylalanine, proline, tryptophan, and valine. Hydrophobic amino acids include glycine, alanine, valine, leucine, isoleucine, proline, phenylalanine, methionine, and tryptophan. Polar amino acids include arginine, asparagine, aspartic acid, glutamine, glutamic acid, histidine, lysine, serine, cysteine, tyrosine, and threonine. The uncharged amino acids include glycine, serine, cysteine, asparagine, glutamine, tyrosine, and threonine, among others.

Thus, as used herein, reference to a polymerase as described herein, such as reference to the amino acid sequence of one or more SEQ ID NOs described herein can include a protein with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference polymerase.

Alternatively, as used herein, reference to a polymerase as described herein, such as reference to the amino acid sequence of one or more SEQ ID NOs described herein can include a protein with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference polymerase.

The present disclosure describes a collection of mutations that result in a polymerase having one or more of the activities described herein. A polymerase described herein can include any number of mutations, e.g., at least 1, at least 2, at least 3, at least 4, at least 5, at least 6, at least 7, at least 8, at least 9, at least 10, at least 11, at least 12, at least 13, at least 14, at least 15, at least 16, at least 17, or at least 18 mutations compared to a reference polymerase, such as SEQ ID NO:1 or SEQ ID NO:8. Likewise, a polymerase described herein can include the mutations in any combination.

In one embodiment, an altered polymerase includes a substitution mutation at a position functionally equivalent to Ala281 in a 9°N polymerase (SEQ ID NO:1). In one embodiment, the substitution mutation at a position functionally equivalent to Ala281 is a mutation to a non-polar, hydrophobic, or uncharged amino acid, for example Gly or Phe.

In one embodiment, an altered polymerase includes a substitution mutation at a position functionally equivalent to Phe283 in a 9°N polymerase (SEQ ID NO:1). In one embodiment, the substitution mutation at a position functionally equivalent to Phe283 is a mutation to a polar or uncharged amino acid, for example Ser.

In one embodiment, an altered polymerase includes a substitution mutation at a position functionally equivalent to Thr349 in a 9°N polymerase (SEQ ID NO:1). In one embodiment, the substitution mutation at a position functionally equivalent to Thr349 is a mutation to a polar or uncharged amino acid, for example Ser, Asn, or Lys.

In one embodiment, an altered polymerase includes a substitution mutation at a position functionally equivalent to Trp397 in a 9°N polymerase (SEQ ID NO:1). In one embodiment, the substitution mutation at a position functionally equivalent to Trp397 is a mutation to a polar or uncharged amino acid, for example Cys.

In one embodiment, an altered polymerase includes a substitution mutation at a position functionally equivalent to Trp397 in a 9°N polymerase (SEQ ID NO:1). In one embodiment, the substitution mutation at a position functionally equivalent to Trp397 is a mutation to a non-polar or hydrophobic amino acid, for example Phe.

In one embodiment, an altered polymerase includes a substitution mutation at a position functionally equivalent to His633 in a 9°N polymerase (SEQ ID NO:1). In one embodiment, the substitution mutation at a position functionally equivalent to His633 is a mutation to a polar or uncharged amino acid, for example Thr.

In one embodiment, an altered polymerase includes at least two, at least three, at least four, or five substitutions mutations at a position functionally equivalent to Ala281, Phe283, Thr349, Trp397, or His633 in a 9°N polymerase (SEQ ID NO:1).

In one embodiment, an altered polymerase includes a substitution mutation at a position functionally equivalent to Arg247 in a 9°N polymerase (SEQ ID NO:1). In one embodiment, the substitution mutation at a position functionally equivalent to Arg247 is a mutation to a polar or uncharged amino acid, for example Tyr.

In one embodiment, an altered polymerase includes a substitution mutation at a position functionally equivalent to Tyr497 in a 9°N polymerase (SEQ ID NO:1). In one embodiment, the substitution mutation at a position functionally equivalent to Tyr497 is a mutation to a non-polar, hydrophobic, or uncharged amino acid, for example Gly.

In one embodiment, an altered polymerase includes a substitution mutation at a position functionally equivalent to Glu599 in a 9°N polymerase (SEQ ID NO:1). In one embodiment, the substitution mutation at a position functionally equivalent to Glu599 is a mutation to a polar amino acid, for example Asp.

In one embodiment, an altered polymerase includes a substitution mutation at a position functionally equivalent to Lys620 in a 9°N polymerase (SEQ ID NO:1). In one embodiment, the substitution mutation at a position functionally equivalent to Lys620 is a mutation to a polar or uncharged amino acid, for example Arg.

In one embodiment, an altered polymerase includes a substitution mutation at a position functionally equivalent to His633 in a 9°N polymerase (SEQ ID NO:1), wherein the mutation is to Gly.

In one embodiment, an altered polymerase includes a substitution mutation at a position functionally equivalent to Val661 in a 9°N polymerase (SEQ ID NO:1). In one embodiment, the substitution mutation at a position functionally equivalent to Val661 is a mutation to a polar or uncharged amino acid, for example Asp.

In one embodiment, an altered polymerase includes at least one, at least two, at least three, at least four, or five substitution mutation at a position functionally equivalent to an amino acid at Ala281, Phe283, Thr349, Trp397, or His633, a substitution mutation at a position functionally equivalent to Tyr497, and at least one, at least two, at least three, at least four, or five substitution mutations at a position functionally equivalent to an amino acid at Arg247, Glu599, Lys620, or Val661 in a 9°N polymerase (SEQ ID NO:1).

In one embodiment, an altered polymerase includes a substitution mutation at a position functionally equivalent to Met129 in a 9°N polymerase (SEQ ID NO:1). In one embodiment, the substitution mutation at a position functionally equivalent to Met129 is a mutation to a non-polar or hydrophobic amino acid, for example Ala.

In one embodiment, an altered polymerase includes a substitution mutation at a position functionally equivalent to Asp141 in a 9°N polymerase (SEQ ID NO:1). In one embodiment, the substitution mutation at a position functionally equivalent to Asp141 is a mutation to a non-polar or hydrophobic amino acid, for example Ala.

In one embodiment, an altered polymerase includes a substitution mutation at a position functionally equivalent to Glu143 in a 9°N polymerase (SEQ ID NO:1). In one embodiment, the substitution mutation at a position functionally equivalent to Glu143 is a mutation to a non-polar or hydrophobic amino acid, for example Ala.

In one embodiment, an altered polymerase includes a substitution mutation at a position functionally equivalent to Cys223 in a 9°N polymerase (SEQ ID NO:1). In one embodiment, the substitution mutation at a position functionally equivalent to Cys223 is a mutation to a polar or uncharged amino acid, for example Ser.

In one embodiment, an altered polymerase includes a substitution mutation at a position functionally equivalent to Lys408 in a 9°N polymerase (SEQ ID NO:1). In one embodiment, the substitution mutation at a position functionally equivalent to Lys408 is a mutation to a non-polar or hydrophobic amino acid, for example Ala.

In one embodiment, an altered polymerase includes a substitution mutation at a position functionally equivalent to Tyr409 in a 9°N polymerase (SEQ ID NO:1). In one embodiment, the substitution mutation at a position functionally equivalent to Tyr409 is a mutation to a non-polar or hydrophobic amino acid, for example Ala.

In one embodiment, an altered polymerase includes a substitution mutation at a position functionally equivalent to Pro410 in a 9°N polymerase (SEQ ID NO:1). In one embodiment, the substitution mutation at a position functionally equivalent to Pro410 is a mutation to a non-polar or hydrophobic amino acid, for example Ile.

In one embodiment, an altered polymerase includes a substitution mutation at a position functionally equivalent to Ala485 in a 9°N polymerase (SEQ ID NO:1). In one embodiment, the substitution mutation at a position functionally equivalent to Ala485 is a mutation to a non-polar or hydrophobic amino acid, for example Val.

In one embodiment, an altered polymerase includes at least one, at least two, at least three, at least four, or five substitution mutation at a position functionally equivalent to an amino acid at Ala281, Phe283, Thr349, Trp397, or His633, a substitution mutation at a position functionally equivalent to Tyr497, at least one, at least two, at least three, at least four, or five substitution mutations at a position functionally equivalent to an amino acid at Arg247, Glu599, His633, Lys620, or Val661, and at least one, at least two, at least three, at least four, at least five, at least six, at least seven, or eight substitution mutation at a position functionally equivalent to amino acid Met129, Asp141, Glu143, Cys223, Lys408, Tyr409, Pro410, or Ala485 in a 9°N polymerase (SEQ ID NO:1).

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to Ala281Gly in the 9°N DNA polymerase amino acid sequence.

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to Ala281Phe in the 9°N DNA polymerase amino acid sequence.

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to Phe283Ser in the 9°N DNA polymerase amino acid sequence.

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to Thr349Ser in the 9°N DNA polymerase amino acid sequence.

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to Thr349Asn in the 9°N DNA polymerase amino acid sequence.

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to Thr349Lys in the 9°N DNA polymerase amino acid sequence.

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to Trp397Cys in the 9°N DNA polymerase amino acid sequence.

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to Trp397Phe in the 9°N DNA polymerase amino acid sequence.

In one embodiment, an altered polymerase includes an amino acid sequence that is at least 80% identical to a 9°N DNA polymerase amino acid sequence SEQ ID NO:8, wherein the polymerase includes an amino acid substitution mutation at a position functionally equivalent to His633Thr in the 9°N DNA polymerase amino acid sequence.

Specific examples of altered polymerases include Pol 1895 (SEQ ID NO:9), Pol 1901 (SEQ ID NO:10), Pol 1920 (SEQ ID NO: 11), Pol 1959 (SEQ ID NO:12), Pol 1962 (SEQ ID NO:13), Pol 1980 (SEQ ID NO:14), Pol 2098 (SEQ ID NO:15), Pol 2139 (SEQ ID NO:16), and Pol 2140 (SEQ ID NO:17).

An altered polymerase described herein can include additional mutations that are known to affect polymerase activity. On such substitution mutation is at a position functionally equivalent to Arg713 in the 9°N polymerase (SEQ ID NO:1). Any of a variety of substitution mutations at one or more of positions known to result in reduced exonuclease activity can be made, as is known in the art and exemplified by U.S. Pat. No. 8,623,628. In one embodiment, the substitution mutation at position Arg713 is a mutation to a non-polar, hydrophobic, or uncharged amino acid, for example Gly, Met, or Ala.

In one embodiment, an altered polymerase includes a substitution mutation at a position functionally equivalent to Arg743 or Lys705, or a combination thereof, in the 9°N polymerase (SEQ ID NO:1), as is known in the art and exemplified by the disclosure of U.S. Pat. No. 8,623,628. In one embodiment, the substitution mutation at position Arg743 or Lys705 is a mutation to a non-polar or hydrophobic amino acid, for example Ala.

The present disclosure also provides compositions that include an altered polymerase described herein. The composition can include other components in addition to the altered polymerase. For example, the composition can include a buffer, a nucleotide solution, or a combination thereof. The nucleotide solution can include nucleotides, such as nucleotides that are labelled, synthetic, modified, or a combination thereof. In one embodiment, a composition includes target nucleic acids, such as a library of target nucleic acids.

### Mutating Polymerases

Various types of mutagenesis are optionally used in the present disclosure, e.g., to modify polymerases to produce variants, e.g., in accordance with polymerase models and model predictions as discussed above, or using random or semi-random mutational approaches. In general, any available mutagenesis procedure can be used for making polymerase mutants. Such mutagenesis procedures optionally include selection of mutant nucleic acids and polypeptides for one or more activity of interest (e.g., reduced pyrophosphorolysis, increased turnover e.g., for a given nucleotide analog). Procedures that can be used include, but are not limited to: site-directed point mutagenesis, random point mutagenesis, in vitro or in vivo homologous recombination (DNA shuffling and combinatorial overlap PCR), mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, degenerate PCR, double-strand break repair, and many others known to persons of skill. The starting polymerase for mutation can be any of those noted herein, including available polymerase mutants such as those identified e.g., in U.S. Pat. Nos. 8,460,910 and 8,623,628, each of which is incorporated by reference in its entirety.

Optionally, mutagenesis can be guided by known information from a naturally occurring polymerase molecule, or of a known altered or mutated polymerase (e.g., using an existing mutant polymerase), e.g., sequence, sequence comparisons, physical properties, crystal structure and/or the like as discussed above. However, in another class of embodiments, modification can be essentially random (e.g., as in classical or “family” DNA shuffling, see, e.g., Crameri et al. (1998) “DNA shuffling of a family of genes from diverse species accelerates directed evolution” Nature 391:288-291).

Additional information on mutation formats is found in: Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2011) (“Ausubel”)) and PCR Protocols A Guide to Methods and Applications (Innis et al. eds) Academic Press Inc. San Diego, Calif (1990) (“Innis”). The following publications and references cited within provide additional detail on mutation formats: Arnold, Protein engineering for unusual environments, Current Opinion in Biotechnology 4:450-455 (1993); Bass et al., Mutant Trp repressors with new DNA-binding specificities, Science 242:240-245 (1988); Bordo and Argos (1991) Suggestions for “Safe” Residue Substitutions in Site-directed Mutagenesis 217:721-729; Botstein & Shortle, Strategies and applications of in vitro mutagenesis, Science 229:1193-1201 (1985); Carter et al., Improved oligonucleotide site-directed mutagenesis using M13 vectors, Nucl. Acids Res. 13: 4431-4443 (1985); Carter, Site-directed mutagenesis, Biochem. J. 237:1-7 (1986); Carter, Improved oligonucleotide-directed mutagenesis using M13 vectors, Methods in Enzymol. 154: 382-403 (1987); Dale et al., Oligonucleotide-directed random mutagenesis using the phosphorothioate method, Methods Mol. Biol. 57:369-374 (1996); Eghtedarzadeh & Henikoff, Use of oligonucleotides to generate large deletions, Nucl. Acids Res. 14: 5115 (1986); Fritz et al., Oligonucleotide-directed construction of mutations: a gapped duplex DNA procedure without enzymatic reactions in vitro, Nucl. Acids Res. 16: 6987-6999 (1988); Grundstrom et al., Oligonucleotide-directed mutagenesis by microscale ‘shot-gun’ gene synthesis, Nucl. Acids Res. 13: 3305-3316 (1985); Hayes (2002) Combining Computational and Experimental Screening for rapid Optimization of Protein Properties PNAS 99(25) 15926-15931; Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel, Rapid and efficient site-specific mutagenesis without phenotypic selection, Proc. Natl. Acad. Sci. USA 82:488-492 (1985); Kunkel et al., Rapid and efficient site-specific mutagenesis without phenotypic selection, Methods in Enzymol. 154, 367-382 (1987); Kramer et al., The gapped duplex DNA approach to oligonucleotide-directed mutation construction, Nucl. Acids Res. 12: 9441-9456 (1984); Kramer & Fritz Oligonucleotide-directed construction of mutations via gapped duplex DNA, Methods in Enzymol. 154:350-367 (1987); Kramer et al., Point Mismatch Repair, Cell 38:879-887 (1984); Kramer et al., Improved enzymatic in vitro reactions in the gapped duplex DNA approach to oligonucleotide-directed construction of mutations, Nucl. Acids Res. 16: 7207 (1988); Ling et al., Approaches to DNA mutagenesis: an overview, Anal Biochem. 254(2): 157-178 (1997); Lorimer and Pastan Nucleic Acids Res. 23, 3067-8 (1995); Mandecki, Oligonucleotide-directed double-strand break repair in plasmids of Escherichia coli: a method for site-specific mutagenesis, Proc. Natl. Acad. Sci. USA, 83:7177-7181(1986); Nakamaye & Eckstein, Inhibition of restriction endonuclease Nci I cleavage by phosphorothioate groups and its application to oligonucleotide-directed mutagenesis, Nucl. Acids Res. 14: 9679-9698 (1986); Nambiar et al., Total synthesis and cloning of a gene coding for the ribonuclease S protein, Science 223: 1299-1301(1984); Sakamar and Khorana, Total synthesis and expression of a gene for the a-subunit of bovine rod outer segment guanine nucleotide-binding protein (transducin), Nucl. Acids Res. 14: 6361-6372 (1988); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Sieber, et al., Nature Biotechnology, 19:456-460 (2001); Smith, In vitro mutagenesis, Ann. Rev. Genet. 19:423-462 (1985); Methods in Enzymol. 100: 468-500 (1983); Methods in Enzymol. 154: 329-350 (1987); Stemmer, Nature 370, 389-91(1994); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Wells et al., Importance of hydrogen-bond formation in stabilizing the transition state of subtilisin, Phil. Trans. R. Soc. Lond. A 317: 415-423 (1986); Wells et al., Cassette mutagenesis: an efficient method for generation of multiple mutations at defined sites, Gene 34:315-323 (1985); Zoller & Smith, Oligonucleotide-directed mutagenesis using M 13-derived vectors: an efficient and general procedure for the production of point mutations in any DNA fragment, Nucleic Acids Res. 10:6487-6500 (1982); Zoller & Smith, Oligonucleotide-directed mutagenesis of DNA fragments cloned into M13 vectors, Methods in Enzymol. 100:468-500 (1983); Zoller & Smith, Oligonucleotide-directed mutagenesis: a simple method using two oligonucleotide primers and a single-stranded DNA template, Methods in Enzymol. 154:329-350 (1987); Clackson et al. (1991) “Making antibody fragments using phage display libraries” Nature 352:624-628; Gibbs et al. (2001) “Degenerate oligonucleotide gene shuffling (DOGS): a method for enhancing the frequency of recombination with family shuffling” Gene 271:13-20; and Hiraga and Arnold (2003) “General method for sequence-independent site-directed chimeragenesis: J. Mol. Biol. 330:287-296. Additional details on many of the above methods can be found in Methods in Enzymology Volume 154, which also describes useful controls for trouble-shooting problems with various mutagenesis methods.

### Making and Isolating Recombinant Polymerases

Generally, nucleic acids encoding a polymerase as presented herein can be made by cloning, recombination, in vitro synthesis, in vitro amplification and/or other available methods. A variety of recombinant methods can be used for expressing an expression vector that encodes a polymerase as presented herein. Methods for making recombinant nucleic acids, expression and isolation of expressed products are well known and described in the art. A number of exemplary mutations and combinations of mutations, as well as strategies for design of desirable mutations, are described herein. Methods for making and selecting mutations in the active site of polymerases, including for modifying steric features in or near the active site to permit improved access by nucleotide analogs are found herein and, e.g., in WO 2007/076057 and WO 2008/051530.

Additional useful references for mutation, recombinant and in vitro nucleic acid manipulation methods (including cloning, expression, PCR, and the like) include Berger and Kimmel, Guide to Molecular Cloning Techniques, Methods in Enzymology volume 152 Academic Press, Inc., San Diego, Calif. (Berger); Kaufman et al. (2003) Handbook of Molecular and Cellular Methods in Biology and Medicine Second Edition Ceske (ed) CRC Press (Kaufman); The Nucleic Acid Protocols Handbook Ralph Rapley (ed) (2000) Cold Spring Harbor, Humana Press Inc (Rapley); Chen et al. (ed) PCR Cloning Protocols, Second Edition (Methods in Molecular Biology, volume 192) Humana Press; and in Viljoen et al. (2005) Molecular Diagnostic PCR Handbook Springer, ISBN 1402034032.

In addition, a plethora of kits are commercially available for the purification of plasmids or other relevant nucleic acids from cells, (see, e.g., EasyPrep™ and FlexiPrep™, both from Pharmacia Biotech; StrataClean™, from Stratagene; and QIAprep™ from Qiagen). Any isolated and/or purified nucleic acid can be further manipulated to produce other nucleic acids, used to transfect cells, incorporated into related vectors to infect organisms for expression, and/or the like. Typical cloning vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both.

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); and Atlas and Parks (eds) The Handbook of Microbiological Media (1993) CRC Press, Boca Raton, Fla.

The present disclosure also includes nucleic acids encoding the altered polymerases disclosed herein. A particular amino acid can be encoded by multiple codons, and certain translation systems (e.g., prokaryotic or eukaryotic cells) often exhibit codon bias, e.g., different organisms often prefer one of the several synonymous codons that encode the same amino acid. As such, nucleic acids presented herein are optionally “codon optimized,” meaning that the nucleic acids are synthesized to include codons that are preferred by the particular translation system being employed to express the polymerase. For example, when it is desirable to express the polymerase in a bacterial cell (or even a particular strain of bacteria), the nucleic acid can be synthesized to include codons most frequently found in the genome of that bacterial cell, for efficient expression of the polymerase. A similar strategy can be employed when it is desirable to express the polymerase in a eukaryotic cell, e.g., the nucleic acid can include codons preferred by that eukaryotic cell.

A variety of protein isolation and detection methods are known and can be used to isolate polymerases, e.g., from recombinant cultures of cells expressing the recombinant polymerases presented herein. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

### Methods of Use

The altered polymerases presented herein can be used in a sequencing procedure, such as a sequencing-by-synthesis (SBS) technique. Briefly, SBS can be initiated by contacting the target nucleic acids with one or more nucleotides (e.g., labelled, synthetic, modified, or a combination thereof), DNA polymerase, etc. Those features where a primer is extended using the target nucleic acid as template will incorporate a labeled nucleotide that can be detected. The incorporation time used in a sequencing run can be significantly reduced using the altered polymerases described herein. Optionally, the labeled nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems, and detection platforms that can be readily adapted for use with an array produced by the methods of the present disclosure are described, for example, in Bentley et al., Nature 456:53-59 (2008); WO 04/018497; WO 91/06678; WO 07/123744; U.S. Pat. Nos. 7,057,026, 7,329,492, 7,211,414, 7,315,019, 7,405,281, and 8,343,746.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320). In pyrosequencing, released PPi can be detected by being converted to adenosine triphosphate (ATP) by ATP sulfurylase, and the resulting ATP can be detected via luciferase-produced photons. Thus, the sequencing reaction can be monitored via a luminescence detection system. Excitation radiation sources used for fluorescence based detection systems are not necessary for pyrosequencing procedures. Useful fluidic systems, detectors and procedures that can be used for application of pyrosequencing to arrays of the present disclosure are described, for example, in WO 2012/058096, US Pat. App. Pub. No. 2005/0191698 A1, U.S. Pat. Nos. 7,595,883 and 7,244,559.

Some embodiments can use methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides. Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008).

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in U.S. Pat. Nos. 8,262,900, 7,948,015, 8,349,167, and US Published Patent Application No. 2010/0137143 A1.

Accordingly, presented herein are methods for incorporating nucleotide analogues into DNA including allowing the following components to interact: (i) an altered polymerase according to any of the above embodiments, (ii) a DNA template; and (iii) a nucleotide solution. In certain embodiments, the DNA template include a clustered array. In certain embodiments, the nucleotides are modified at the 3′ sugar hydroxyl, and include modifications at the 3′ sugar hydroxyl such that the substituent is larger in size than the naturally occurring 3′ hydroxyl group.

### Nucleic Acids Encoding Altered Polymerases

The present disclosure also includes nucleic acid molecules encoding the altered polymerases described herein. For any given altered polymerase which is a mutant version of a polymerase for which the amino acid sequence and preferably also the wild type nucleotide sequence encoding the polymerase is known, it is possible to obtain a nucleotide sequence encoding the mutant according to the basic principles of molecular biology. For example, given that the wild type nucleotide sequence encoding 9°N polymerase is known, it is possible to deduce a nucleotide sequence encoding any given mutant version of 9°N having one or more amino acid substitutions using the standard genetic code. Similarly, nucleotide sequences can readily be derived for mutant versions other polymerases such as, for example, Vent® polymerase, Deep Vent® polymerase, Pfu polymerase, KOD polymerase, Pab polymerase, etc. Nucleic acid molecules having the required nucleotide sequence may then be constructed using standard molecular biology techniques known in the art.

In accordance with the embodiments presented herein, a defined nucleic acid includes not only the identical nucleic acid but also any minor base variations including, in particular, substitutions in cases which result in a synonymous codon (a different codon specifying the same amino acid residue) due to the degenerate code in conservative amino acid substitutions. The term “nucleic acid sequence” also includes the complementary sequence to any single stranded sequence given regarding base variations.

The nucleic acid molecules described herein may also, advantageously, be included in a suitable expression vector to express the polymerase proteins encoded therefrom in a suitable host. Incorporation of cloned DNA into a suitable expression vector for subsequent transformation of said cell and subsequent selection of the transformed cells is well known to those skilled in the art as provided in Sambrook et al. (1989), Molecular cloning: A Laboratory Manual, Cold Spring Harbor Laboratory.

Such an expression vector includes a vector having a nucleic acid according to the embodiments presented herein operably linked to regulatory sequences, such as promoter regions, that are capable of effecting expression of said DNA fragments. The term “operably linked” refers to a juxtaposition wherein the components described are in a relationship permitting them to function in their intended manner. Such vectors may be transformed into a suitable host cell to provide for the expression of a protein according to the embodiments presented herein.

The nucleic acid molecule may encode a mature protein or a protein having a pro-sequence, including that encoding a leader sequence on the preprotein which is then cleaved by the host cell to form a mature protein. The vectors may be, for example, plasmid, virus or phage vectors provided with an origin of replication, and optionally a promoter for the expression of said nucleotide and optionally a regulator of the promoter. The vectors may contain one or more selectable markers, such as, for example, an antibiotic resistance gene.

Regulatory elements required for expression include promoter sequences to bind RNA polymerase and to direct an appropriate level of transcription initiation and also translation initiation sequences for ribosome binding. For example, a bacterial expression vector may include a promoter such as the lac promoter and for translation initiation the Shine-Dalgarno sequence and the start codon AUG. Similarly, a eukaryotic expression vector may include a heterologous or homologous promoter for RNA polymerase II, a downstream polyadenylation signal, the start codon AUG, and a termination codon for detachment of the ribosome. Such vectors may be obtained commercially or be assembled from the sequences described by methods well known in the art.

Transcription of DNA encoding the polymerase by higher eukaryotes may be optimized by including an enhancer sequence in the vector. Enhancers are cis-acting elements of DNA that act on a promoter to increase the level of transcription. Vectors will also generally include origins of replication in addition to the selectable markers.

The present disclosure also provides a kit for performing a nucleotide incorporation reaction. The kit includes at least one altered polymerase described herein and a nucleotide solution in a suitable packaging material in an amount sufficient for at least one nucleotide incorporation reaction. Optionally, other reagents such as buffers and solutions needed to use the altered polymerase and nucleotide solution are also included. Instructions for use of the packaged components are also typically included.

In certain embodiments, the nucleotide solution includes labelled nucleotides. In certain embodiments, the nucleotides are synthetic nucleotides. In certain embodiments, the nucleotides are modified nucleotides. In certain embodiments, a modified nucleotide has been modified at the 3′ sugar hydroxyl such that the substituent is larger in size than the naturally occurring 3′ hydroxyl group. In certain embodiments, the modified nucleotides include a modified nucleotide or nucleoside molecule that includes a purine or pyrimidine base and a ribose or deoxyribose sugar moiety having a removable 3′-OH blocking group covalently attached thereto, such that the 3′ carbon atom has attached a group of the structure

—O—Z
wherein Z is any of —C(R′)₂—O—R″, —C(R′)₂—N(R″)₂, —C(R′)₂—N(H)R″, —C(R′)₂—S—R″ and —C(R′)₂—F,
wherein each R″ is or is part of a removable protecting group;
each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; or (R′)₂ represents an alkylidene group of formula=C(R′″)₂ wherein each R′″ may be the same or different and is selected from the group comprising hydrogen and halogen atoms and alkyl groups; and
wherein the molecule may be reacted to yield an intermediate in which each R″ is exchanged for H or, where Z is —C(R′)₂—F, the F is exchanged for OH, SH or NH₂, preferably OH, which intermediate dissociates under aqueous conditions to afford a molecule with a free 3′OH;
with the proviso that where Z is —C(R′)₂-S—R″, both R′ groups are not H. In certain embodiments, R′ of the modified nucleotide or nucleoside is an alkyl or substituted alkyl. In certain embodiments, —Z of the modified nucleotide or nucleoside is of formula —C(R′)₂—N₃. In certain embodiments, Z is an azidomethyl group.

In certain embodiments, the modified nucleotides are fluorescently labelled to allow their detection. In certain embodiments, the modified nucleotides include a nucleotide or nucleoside having a base attached to a detectable label via a cleavable linker. In certain embodiments, the detectable label includes a fluorescent label.

As used herein, the phrase “packaging material” refers to one or more physical structures used to house the contents of the kit. The packaging material is constructed by known methods, preferably to provide a sterile, contaminant-free environment. The packaging material has a label which indicates that the components can be used for conducting a nucleotide incorporation reaction. In addition, the packaging material contains instructions indicating how the materials within the kit are employed to practice a nucleotide incorporation reaction. As used herein, the term “package” refers to a solid matrix or material such as glass, plastic, paper, foil, and the like, capable of holding within fixed limits the polypeptides. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The complete disclosure of the patents, patent documents, and publications cited in the Background, the Detailed Description of Exemplary Embodiments, and elsewhere herein are incorporated by reference in their entirety as if each were individually incorporated.

Illustrative embodiments of this invention are discussed, and reference has been made to possible variations within the scope of this invention. These and other variations, combinations, and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

### EXEMPLARY EMBODIMENTS

Embodiment 1. A recombinant DNA polymerase comprising an amino acid sequence that is at least 80% identical to a 9°N DNA polymerase amino acid sequence SEQ ID NO:1, wherein the DNA polymerase comprises an amino acid substitution mutation at a position functionally equivalent to Ala281, Phe283, Thr349, or Trp397 in the 9°N DNA polymerase amino acid sequence.

Embodiment 2. The polymerase of Embodiment 1, wherein the substitution mutation at the position functionally equivalent to Ala281 comprises a mutation to a non-polar, hydrophobic, or uncharged amino acid.

Embodiment 3. The polymerase of any one of Embodiments 1-2, wherein the substitution mutation at the position functionally equivalent to Ala281 comprises a mutation to Gly or Phe.

Embodiment 4. The polymerase of any one of Embodiments 1-3, wherein the substitution mutation at the position functionally equivalent to Phe283 comprises a mutation to a polar of uncharged amino acid.

Embodiment 5. The polymerase of any one of Embodiments 1-4, wherein the substitution mutation at the position functionally equivalent to Phe283 comprises a mutation to Ser.

Embodiment 6. The polymerase of any one of Embodiments 1-5, wherein the substitution mutation at the position functionally equivalent to Thr349 comprises a mutation to a polar or uncharged amino acid.

Embodiment 7. The polymerase of any one of Embodiments 1-6, wherein the substitution mutation at the position functionally equivalent to Thr349 comprises a mutation to Ser or Asn.

Embodiment 8. The polymerase of any one of Embodiments 1-7, wherein the substitution mutation at the position functionally equivalent to Thr349 comprises a mutation to a charged amino acid.

Embodiment 9. The polymerase of any one of Embodiments 1-8, wherein the substitution mutation at the position functionally equivalent to Thr349 comprises a mutation to Lys.

Embodiment 10. The polymerase of any one of Embodiments 1-9, wherein the substitution mutation at the position functionally equivalent to Trp397 comprises a mutation to a polar or uncharged amino acid.

Embodiment 11. The polymerase of any one of Embodiments 1-10, wherein the substitution mutation at the position functionally equivalent to Trp397 comprises a mutation to Cys.

Embodiment 12. The polymerase of any one of Embodiments 1-11, wherein the substitution mutation at the position functionally equivalent to Trp397 comprises a mutation to a non-polar or hydrophobic amino acid.

Embodiment 13. The polymerase of any one of Embodiments 1-12, wherein the substitution mutation at the position functionally equivalent to Trp397 comprises a mutation to Phe.

Embodiment 14. The polymerase of any one of Embodiments 1-13, wherein the polymerase comprises at least two, at least three, or four amino acid substitution mutations at positions functionally equivalent to an amino acid selected from A281, F283, Thr349, or Trp397 in the 9°N DNA polymerase amino acid sequence.

Embodiment 15. The polymerase of any one of Embodiments 1-14, wherein the polymerase comprises the substitution mutation at the position functionally equivalent to Thr349, and further comprises amino acid substitution mutations at positions functionally equivalent to amino acids Met129, Asp141, Glu143, Cys223, Leu408, Tyr409, Pro410, Ala485, Tyr497, Arg247, Glu599, and His633 in the 9°N DNA polymerase amino acid sequence.

Embodiment 16. The polymerase of any one of Embodiments 1-15, wherein the substitution mutation at the position functionally equivalent to Thr349 comprises a mutation to a charged amino acid.

Embodiment 17. The polymerase of any one of Embodiments 1-16, wherein the substitution mutation at the position functionally equivalent to Thr349 comprises a mutation to Lys.

Embodiment 18. The polymerase of any one of Embodiments 1-17, wherein the substitution mutation at the position functionally equivalent to Met129 comprises a mutation to a non-polar or hydrophobic amino acid.

Embodiment 19. The polymerase of any one of Embodiments 1-18, wherein the substitution mutation at the position functionally equivalent to Met129 comprises a mutation to Ala.

Embodiment 20. The polymerase of any one of Embodiments 1-19, wherein the substitution mutation at the position functionally equivalent to Asp141 comprises a mutation to a non-polar or hydrophobic amino acid.

Embodiment 21. The polymerase of any one of Embodiments 1-20, wherein the substitution mutation at the position functionally equivalent to Asp141 comprises a mutation to Ala.

Embodiment 22. The polymerase of any one of Embodiments 1-21, wherein the substitution mutation at the position functionally equivalent to Glu143 comprises a mutation to a non-polar or hydrophobic amino acid.

Embodiment 23. The polymerase of any one of Embodiments 1-22, wherein the substitution mutation at the position functionally equivalent to Glu143 comprises a mutation to Ala.

Embodiment 24. The polymerase of any one of Embodiments 1-23, wherein the substitution mutation at the position functionally equivalent to Cys223 comprises a mutation to a polar or uncharged amino acid.

Embodiment 25. The polymerase of any one of Embodiments 1-24, wherein the substitution mutation at the position functionally equivalent to Cys223 comprises a mutation to Ser.

Embodiment 26. The polymerase of any one of Embodiments 1-25, wherein the substitution mutation at the position functionally equivalent to Leu408 comprises a mutation to a non-polar or hydrophobic amino acid.

Embodiment 27. The polymerase of any one of Embodiments 1-26, wherein the substitution mutation at the position functionally equivalent to Leu408 comprises a mutation to Ala.

Embodiment 28. The polymerase of any one of Embodiments 1-27, wherein the substitution mutation at the position functionally equivalent to Tyr409 comprises a mutation to a non-polar or hydrophobic amino acid.

Embodiment 29. The polymerase of any one of Embodiments 1-28, wherein the substitution mutation at the position functionally equivalent to Tyr409 comprises a mutation to Ala.

Embodiment 30. The polymerase of any one of Embodiments 1-29, wherein the substitution mutation at the position functionally equivalent to Pro410 comprises a mutation to a non-polar or hydrophobic amino acid.

Embodiment 31. The polymerase of any one of Embodiments 1-30, wherein the substitution mutation at the position functionally equivalent to Pro410 comprises a mutation to Ile.

Embodiment 32. The polymerase of any one of Embodiments 1-31, wherein the substitution mutation at the position functionally equivalent to Ala485 comprises a mutation to a non-polar or hydrophobic amino acid.

Embodiment 33. The polymerase of any one of Embodiments 1-32, wherein the substitution mutation at the position functionally equivalent to Ala485 comprises a mutation to Val.

Embodiment 34. The polymerase of any one of Embodiments 1-33, wherein the substitution mutation at the position functionally equivalent to Tyr497 comprises a mutation to a non-polar, hydrophobic, or uncharged amino acid.

Embodiment 35. The polymerase of any one of Embodiments 1-34, wherein the substitution mutation at the position functionally equivalent to Tyr497 comprises a mutation to Gly.

Embodiment 36. The polymerase of any one of Embodiments 1-35, wherein the substitution mutation at the position functionally equivalent to Arg247 comprises a mutation to a polar or uncharged amino acid.

Embodiment 37. The polymerase of any one of Embodiments 1-36, wherein the substitution mutation at the position functionally equivalent to Arg247 comprises a mutation to Tyr.

Embodiment 38. The polymerase of any one of Embodiments 1-37, wherein the substitution mutation at the position functionally equivalent to Glu599 comprises a mutation to a polar amino acid.

Embodiment 39. The polymerase of any one of Embodiments 1-38, wherein the substitution mutation at the position functionally equivalent to Glu599 comprises a mutation to Asp.

Embodiment 40. The polymerase of any one of Embodiments 1-39, wherein the substitution mutation at the position functionally equivalent to His633 comprises a mutation to a non-polar, hydrophobic, or uncharged amino acid.

Embodiment 41. The polymerase of any one of Embodiments 1-40, wherein the substitution mutation at the position functionally equivalent to His633 comprises a mutation to Gly.

Embodiment 42. The polymerase of any one of Embodiments 1-41, further comprising an amino acid substitution mutation at a position functionally equivalent to Tyr497 and at least one amino acid substitution mutation at a position functionally equivalent to Arg247, Glu599, or His633 in the 9°N DNA polymerase amino acid sequence.

Embodiment 43. The polymerase of any one of Embodiments 1-42, wherein the substitution mutation at the position functionally equivalent to Tyr497 comprises a mutation to a non-polar, hydrophobic, or uncharged amino acid.

Embodiment 44. The polymerase of any one of Embodiments 1-43, wherein the substitution mutation at the position functionally equivalent to Tyr497 comprises a mutation to Gly.

Embodiment 45. The polymerase of any one of Embodiments 1-44, wherein the substitution mutation at the position functionally equivalent to Arg247 comprises a mutation to a polar or uncharged amino acid.

Embodiment 46. The polymerase of any one of Embodiments 1-45, wherein the substitution mutation at the position functionally equivalent to Arg247 comprises a mutation to Tyr.

Embodiment 47. The polymerase of any one of Embodiments 1-46, wherein the substitution mutation at the position functionally equivalent to Glu599 comprises a mutation to a polar amino acid.

Embodiment 48. The polymerase of any one of Embodiments 1-47, wherein the substitution mutation at the position functionally equivalent to Glu599 comprises a mutation to Asp.

Embodiment 49. The polymerase of any one of Embodiments 1-48, wherein the substitution mutation at the position functionally equivalent to His633 comprises a mutation to a non-polar, hydrophobic, or uncharged amino acid.

Embodiment 50. The polymerase of any one of Embodiments 1-49, wherein the substitution mutation at the position functionally equivalent to His633 comprises a mutation to Gly.

Embodiment 51. The polymerase of any one of Embodiments 1-50, wherein the polymerase comprises an amino acid substitution mutation at a position functionally equivalent to Tyr497 and at least two or three amino acid substitution mutations at positions functionally equivalent to an amino acid selected from Arg247, Glu599, or His633 in the 9°N DNA polymerase amino acid sequence.

Embodiment 52. The polymerase of any one of Embodiments 1-51, further comprising at least one amino acid substitution mutation at a position functionally equivalent to Lys620 or Val661 in the 9°N DNA polymerase amino acid sequence.

Embodiment 53. The polymerase of any one of Embodiments 1-52, wherein the substitution mutation at the position functionally equivalent to Lys620 comprises a mutation to a polar amino acid.

Embodiment 54. The polymerase of any one of Embodiments 1-53, wherein the substitution mutation at the position functionally equivalent to Lys620 comprises a mutation to Arg.

Embodiment 55. The polymerase of any one of Embodiments 1-53, wherein the substitution mutation at the position functionally equivalent to Val661 comprises a mutation to a polar amino acid.

Embodiment 56. The polymerase of any one of Embodiments 1-55, wherein the substitution mutation at the position functionally equivalent to Val661 comprises a mutation to Asp.

Embodiment 57. The polymerase of any one of Embodiments 1-56, wherein the polymerase comprises an amino acid substitution mutation at a position functionally equivalent to Tyr497 and at least two, at least three, at least four, or at least five amino acid substitution mutations at positions functionally equivalent to an amino acid selected from Arg247, Glu599, His633, Lys620, or Val661 in the 9°N DNA polymerase amino acid sequence.

Embodiment 58. The polymerase of any one of Embodiments 1-57, wherein the polymerase further comprises amino acid substitution mutations at positions functionally equivalent to amino acids Met129, Asp141, Glu143, Cys223, Leu408, Tyr409, Pro410, or Ala485 in the 9°N DNA polymerase amino acid sequence.

Embodiment 59. A recombinant DNA polymerase comprising an amino acid sequence that is at least 80% identical to a 9°N DNA polymerase amino acid sequence SEQ ID NO:8, wherein the DNA polymerase comprises an amino acid substitution mutation at a position functionally equivalent to Ala281Gly in the 9°N DNA polymerase amino acid sequence.

Embodiment 60. A recombinant DNA polymerase comprising an amino acid sequence that is at least 80% identical to a 9°N DNA polymerase amino acid sequence SEQ ID NO: 8, wherein the DNA polymerase comprises an amino acid substitution mutation at a position functionally equivalent to Ala281Phe in the 9°N DNA polymerase amino acid sequence.

Embodiment 61. A recombinant DNA polymerase comprising an amino acid sequence that is at least 80% identical to a 9°N DNA polymerase amino acid sequence SEQ ID NO: 8, wherein the DNA polymerase comprises an amino acid substitution mutation at a position functionally equivalent to Phe283Ser in the 9°N DNA polymerase amino acid sequence.

Embodiment 62. A recombinant DNA polymerase comprising an amino acid sequence that is at least 80% identical to a 9°N DNA polymerase amino acid sequence SEQ ID NO: 8, wherein the DNA polymerase comprises an amino acid substitution mutation at a position functionally equivalent to Thr349Ser in the 9°N DNA polymerase amino acid sequence.

Embodiment 63. A recombinant DNA polymerase comprising an amino acid sequence that is at least 80% identical to a 9°N DNA polymerase amino acid sequence SEQ ID NO: 8, wherein the DNA polymerase comprises an amino acid substitution mutation at a position functionally equivalent to Thr349Asn in the 9°N DNA polymerase amino acid sequence.

Embodiment 64. A recombinant DNA polymerase comprising an amino acid sequence that is at least 80% identical to a 9°N DNA polymerase amino acid sequence SEQ ID NO: 8, wherein the DNA polymerase comprises an amino acid substitution mutation at a position functionally equivalent to Thr349Lys in the 9°N DNA polymerase amino acid sequence.

Embodiment 65. A recombinant DNA polymerase comprising an amino acid sequence that is at least 80% identical to a 9°N DNA polymerase amino acid sequence SEQ ID NO: 8, wherein the DNA polymerase comprises an amino acid substitution mutation at a position functionally equivalent to Trp397Cys in the 9°N DNA polymerase amino acid sequence.

Embodiment 66. A recombinant DNA polymerase comprising an amino acid sequence that is at least 80% identical to a 9°N DNA polymerase amino acid sequence SEQ ID NO: 8, wherein the DNA polymerase comprises an amino acid substitution mutation at a position functionally equivalent to Trp397Phe in the 9°N DNA polymerase amino acid sequence.

Embodiment 67. A recombinant DNA polymerase comprising an amino acid sequence that is at least 80% identical to a 9°N DNA polymerase amino acid sequence SEQ ID NO: 8, wherein the DNA polymerase comprises an amino acid substitution mutation at a position functionally equivalent to His633Thr in the 9°N DNA polymerase amino acid sequence.

Embodiment 68. A DNA polymerase comprising the amino acid sequence of any one of SEQ ID NOs:9-17.

Embodiment 69. The polymerase of any one of Embodiments 1-68, wherein the polymerase is a family B type DNA polymerase.

Embodiment 70. The polymerase of any one of Embodiments 1-69, wherein the polymerase is selected from the group consisting of a family B archaeal DNA polymerase, a human DNA polymerase-a, T4 polymerase, RB69 polymerase, and phi29 phage DNA polymerase.

Embodiment 71. The polymerase of any one of Embodiments 1-70, wherein the family B archaeal DNA polymerase is from a genus selected from the group consisting of Thermococcus, Pyrococcus, and Methanococcus.

Embodiment 72. The polymerase of any of any one of Embodiments 1-71, wherein the polymerase comprises reduced exonuclease activity as compared to a wild type polymerase.

Embodiment 73. A nucleic acid molecule encoding a polymerase as defined in any one of any one of Embodiments 1-72.

Embodiment 74. An expression vector comprising the nucleic acid molecule of Embodiment 73.

Embodiment 75. A host cell comprising the vector of Embodiment 74.

Embodiment 76. A method for incorporating modified nucleotides into DNA comprising allowing the following components to interact: (i) a polymerase according to any one of any one of Embodiments 1-72, (ii) a DNA template; and (iii) a nucleotide solution.

Embodiment 77. The method of Embodiment 76, wherein the DNA template comprises a clustered array.

Embodiment 78. A kit for performing a nucleotide incorporation reaction comprising: a polymerase as defined in any one of any one of Embodiments 1-72 and a nucleotide solution.

Embodiment 79. The kit of any one of Embodiment 78, wherein the nucleotide solution comprises labelled nucleotides.

Embodiment 80. The kit of any one of Embodiments 78-79, wherein the nucleotides comprise synthetic nucleotides.

Embodiment 81. The kit of any one of Embodiments 78-80, wherein the nucleotides comprise modified nucleotides.

Embodiment 82. The kit of any one of Embodiments 78-81, wherein the modified nucleotides have been modified at the 3′ sugar hydroxyl such that the substituent is larger in size than the naturally occurring 3′ hydroxyl group.

Embodiment 83. The kit of any one of Embodiments 78-82, wherein the modified nucleotides comprise a modified nucleotide or nucleoside molecule comprising a purine or pyrimidine base and a ribose or deoxyribose sugar moiety having a 7 removable 3′-OH blocking group covalently attached thereto, such that the 3′ carbon atom has attached a group of the structure

—O—Z
wherein Z is any of —C(R′)₂—O—R″, —C(R′)₂—N(R″)₂, —C(R′)₂—N(H)R″, —C(R′)₂—S—R″ and —C(R′)₂—F, wherein each R″ is or is part of a removable protecting group;
each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; or (R′)₂ represents an alkylidene group of formula=C(R′″)₂ wherein each R′″ may be the same or different and is selected from the group comprising hydrogen and halogen atoms and alkyl groups; and
wherein said molecule may be reacted to yield an intermediate in which each R″ is exchanged for H or, where Z is —C(R′)₂—F, the F is exchanged for OH, SH or NH₂, preferably OH, which intermediate dissociates under aqueous conditions to afford a molecule with a free 3′OH;
with the proviso that where Z is —C(R′)₂-S—R″, both R′ groups are not H.

Embodiment 84. The kit of any one of Embodiments 78-83, wherein R′ of the modified nucleotide or nucleoside is an alkyl or substituted alkyl.

Embodiment 85. The kit of any one of Embodiments 78-84, wherein —Z of the modified nucleotide or nucleoside is of formula —C(R′)₂-N₃.

Embodiment 86. The kit of any one of Embodiments 78-85, wherein Z is an azidomethyl group.

87. The kit of any one of Embodiments 78-86, wherein the modified nucleotides are fluorescently labelled to allow their detection.

88. The kit of any one of Embodiments 78-86, wherein the modified nucleotides comprise a nucleotide or nucleoside having a base attached to a detectable label via a cleavable linker.

89. The kit of any one of Embodiments 78-88, wherein the detectable label comprises a fluorescent label.

90. The kit of any one of Embodiments 78-88, further comprising one or more DNA template molecules and/or primers.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

### General Assay Methods and Conditions

Unless otherwise noted, this describes the general assay conditions used in the Examples described herein.

### A. Cloning and Expression of Polymerases

Methods for making recombinant nucleic acids, expression, and isolation of expressed products are known and described in the art. Mutagenesis was performed on the coding region encoding a 9°N polymerase (SEQ ID NO:1) using standard site-directed mutagenesis methodology. PCR-based approaches were used to amplify mutated coding regions and add a His-tag. For each mutation made, the proper sequence of the altered coding region was confirmed by determining the sequence of the cloned DNA.

His-tagged mutant polymerase coding regions were subcloned into pET11a vector and transformed into BL21 Star (DE3) expression cells (Invitrogen). Overnight cultures from single-picked colonies were used to inoculate expression cultures in 2.8L flasks. Cultures were grown at 37° C. until OD600 of about 0.8, protein expression was then induced with 0.2 mM IPTG and followed by 4 hours of additional growth. Cultures were centrifuged at 7000 rpm for 20 minutes. Cell pellets were stored at −20° C. until purification.

Pellets were freeze-thawed and lysed with 5× w/v lysis buffer (50 mM Tris-HCl pH7.5, 1 mM EDTA, 0.1% BME, and 5% Glycerol) in the presence of Ready-Lyse and Omnicleave reagents (Epicentre) according to manufacturer recommendations. The final NaCl concentration was raised to 500 mM and lysate was incubated on ice for 5 minutes. Following centrifugation, the supernatant was incubated at 80° C. for about 70 minutes. All further purification was performed at 4° C. Supernatant was iced for 30 min before being centrifuged and purified using 5 mL Ni Sepharose HP columns (GE). Columns were pre-equilibrated with Buffer A (50 mM Tris-HCl pH 7.5, 1 mM EDTA, 5% Glycerol, 500 mM NaCl, and 20 mM Imidazole). The column was eluted using a 75 mL gradient from 20 to 500 mM imidazole. Peak fractions were pooled and diluted with 10% glycerol to match the conductivity of SP Buffer A (50 mM Tris-HCl pH 7.5, 150 mM NaCl, 1 mM EDTA, 5% Glycerol) and loaded onto 5 mL SP Sepharose columns (GE). The column was eluted using a 100 mL gradient from 150 to 1000 mM NaCl. Peak fractions were pooled, dialyzed into storage buffer (10 mM Tris-HCl pH 7.5, 300 mM KCL, 0.1 mM EDTA, and 50% Glycerol) and stored at −20° C.

### B. Error Rate and Phasing Analysis

Sequencing experiments were used to compare error rates and phasing values. Unless indicated otherwise, the experiments were carried out on a MiniSeq™ system (Illumina, Inc., San Diego, Calif), according to manufacturer instructions. For example, for each polymerase, a separate incorporation mix (IMX) was prepared and used in a short run (35 cycles in read 1) or long run (227 cycle run of 151 in read 1 and 76 in read 2). Standard MiniSeq Mid Output Reagent Cartridge formulations were used, with the standard polymerase substituted with the polymerase being tested, at a concentration of 90 μg/mL. The time for incubation of IMX on the flowcell varied as noted in the Examples herein. The DNA library used was made following the standard TruSeq™ Nano protocol (Illumina, Inc.), with 350 bp target insert size, using E. coli genomic DNA; PhiX DNA (Illumina, Inc) was added to resulting library in ˜1:10 molar ratio. Illumina RTA Software was used to evaluate error rate on both genomes as well as phasing levels.

### Example 2

### Sequencing Performance of Selected Altered Polymerases

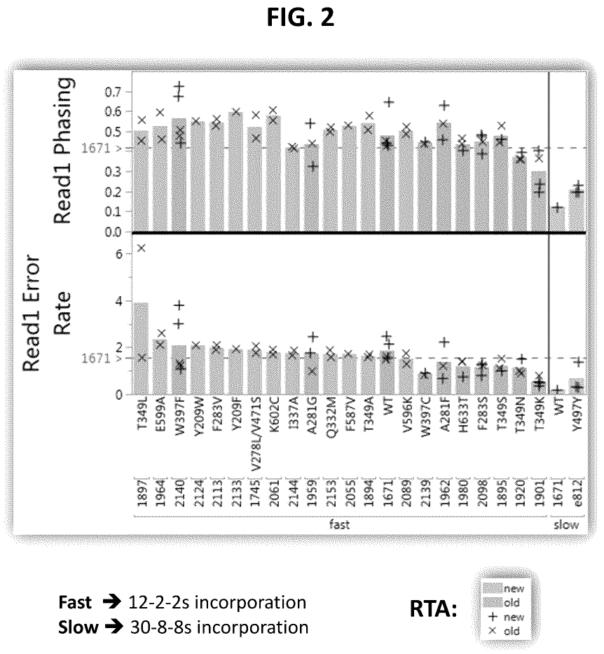

A number of altered polymerases were identified that had error rates and phasing levels in a short run under a short incorporation time (e.g., 16 sec) that were not significantly greater than a control polymerase used in a short run under a standard incorporation time (46 sec). The quality metrics used to evaluate the altered polymerases were the phasing rates (“Read1 Phasing”) and cumulative error rates of E. coli and bacteriophage PhiX sequencing controls (“Read1 Error”). The quality metrics were compared to corresponding Pol 1671 and Pol 812 phasing and error rates at the standard incorporation time during long sequencing runs (227 cycles of 151 in read 1 and 76 in read 2). Results are summarized in FIG. 2.

FIG. 2 shows the error rates and phasing levels of selected altered polymerases of the present disclosure. Starting at the bottom of the figure, “slow” refers to slow incorporation time, i.e., the standard incorporation time of 46 seconds; “fast” refers to the faster incorporation time of 16 seconds; “1671”, “1901”, etc., identify specific altered polymerases; WT, T349K, T349N, T349S, etc., refer to specific mutations in an altered polymerase relative to Pol 1671 (“WT” corresponds to Pol 1671 for the purposes of this application); and Pols 1671 and e812 refer to control polymerases.

A number of newly identified mutants, e.g., Pols 1895, 1901, 1920, 1962, 1980, 2089, 2098, and 2139, demonstrated lower error rates under a short incorporation time than the Pol 1671 control without a significant increase in phasing. Notably, Pols 1901 and 1920 showed both lower phasing and cumulative error rates relative to the Pol 1671 standard. These altered polymerases are characterized by substitutions at position 349, wherein a threonine is replaced with a lysine (Pol 1901) or asparagine (Pol 1920).

### Example 3

### Superior Sequencing Performance of Pol 1901

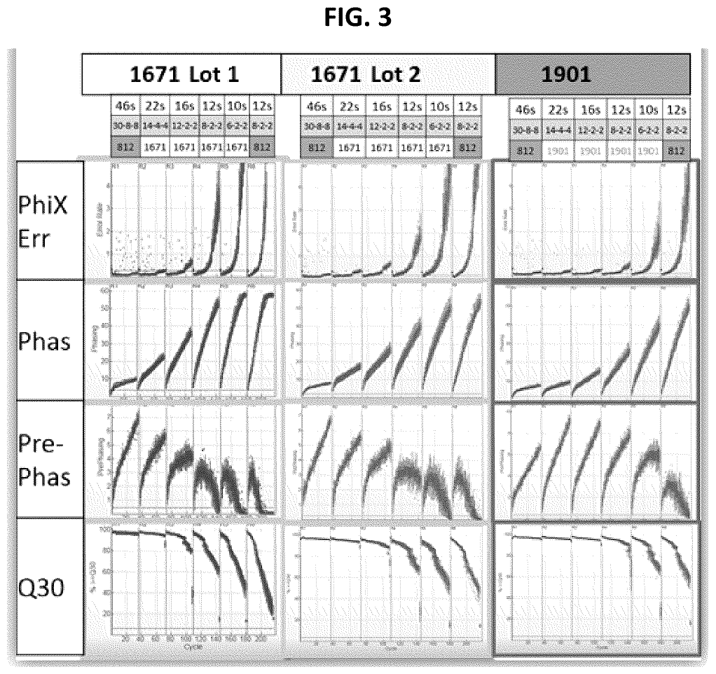

To identify the limits of polymerase performance, two different lots of Pol 1671 were compared to Pol 1901 at different incorporation speeds throughout a series of 35-cycle sequencing runs. The quality metrics used to compare the altered polymerases were the cumulative error rate, phasing, pre-phasing, and % Q30. Pol 812 was once again used as a standard. Results are summarized in FIG. 3.

In the first row of FIG. 3, “46s”, “22s”, “16s”, “12s”, and “10s” refer to the total incorporation time. In the second row, “30-8-8”, “14-4-4”, etc. refer to the static incubation period (in seconds) followed by the two mixing periods (also in seconds). The third row refers to the identities of the altered polymerases. “PhiX Err” refers to the observed error rates; “Phas” refers to the observed phasing values; “Pre-Phas” refers to the observed pre-phasing values; and “Q30” refers to the percentage of reads that pass the Q30 quality filter, i.e. an error rate of less of equal to 1 in 1000, or 0.1%.

Referring to the PhiX error rates, FIG. 3 shows that Pol 1901 maintained a low error rate at as low as 12 sec total incorporation time, whereas the Pol 1671 error rate increased dramatically below 16 sec incorporation time. Similar improvements in Pol 1901 over Pol 1671 were observed at 12 sec total incorporation time in the phasing, pre-phasing and % Q30 sequencing quality metrics.

The complete disclosure of all patents, patent applications (whether published or not), and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Any patent, patent application (whether published or not), or other literature referred to herein in hereby incorporated herein in its respective entirety or in part to the extent that it does not conflict with the disclosure presented herein.

In addition to the documents already cited in this application, reference is hereby made to provisional U.S. Patent Application No. 62/753,558, identically entitled “Polymerases, compositions, and methods of use,” filed Oct. 31, 2018. The entire contents of this application is also incorporated herein by reference.

1. A recombinant DNA polymerase comprising an amino acid sequence that is at least 80% identical to a polymerase amino acid sequence SEQ ID NO:4, SEQ ID NO:5, or SEQ ID NO:6, wherein the DNA polymerase comprises an amino acid substitution mutation at a position functionally equivalent to Ala281, Phe283, Thr349, or Trp397 in the 9°N DNA polymerase amino acid sequence SEQ ID NO:1.

2. (canceled)

3. The polymerase of claim 1, wherein the substitution mutation at the position functionally equivalent to Ala281 comprises a mutation to Gly or Phe.

4. (canceled)

5. The polymerase of claim 1, wherein the substitution mutation at the position functionally equivalent to Phe283 comprises a mutation to Ser.

6. (canceled)

7. The polymerase of claim 1, wherein the substitution mutation at the position functionally equivalent to Thr349 comprises a mutation to Ly Ser or Asn.

8. (canceled)

9. (canceled)

10. (canceled)

11. The polymerase of claim 1, wherein the substitution mutation at the position functionally equivalent to Trp397 comprises a mutation to Cys or Phe.

12. (canceled)

13. (canceled)

14. The polymerase of claim 1, wherein the polymerase comprises at least two, at least three, or four amino acid substitution mutations at positions functionally equivalent to an amino acid selected from A281, F283, Thr349, or Trp397 in the 9°N DNA polymerase amino acid sequence SEQ ID NO:1.

15. The polymerase of claim 1, wherein the polymerase comprises the substitution mutation at the position functionally equivalent to Thr349, and further comprises amino acid substitution mutations at positions functionally equivalent to amino acids Met129, Asp141, Glu143, Cys223, Leu408, Tyr409, Pro410, Ala485, Tyr497, Arg247, Glu599, and His633 in the 9°N DNA polymerase amino acid sequence SEQ ID NO:1.

16. (canceled)

17. The polymerase of claim 1, wherein the substitution mutation at the position functionally equivalent to Thr349 comprises a mutation to Lys.

18. (canceled)

19. The polymerase of claim 1, wherein the substitution mutation at the position functionally equivalent to Met129 comprises a mutation to Ala.

20. (canceled)

21. The polymerase of claim 1, wherein the substitution mutation at the position functionally equivalent to Asp141 comprises a mutation to Ala.

22. (canceled)

23. The polymerase of claim 1, wherein the substitution mutation at the position functionally equivalent to Glu143 comprises a mutation to Ala.

24. (canceled)

25. The polymerase of claim 1, wherein the substitution mutation at the position functionally equivalent to Cys223 comprises a mutation to Ser.

26. (canceled)

27. The polymerase of claim 15, wherein the substitution mutation at the position functionally equivalent to Leu408 comprises a mutation to Ala.

28. (canceled)

29. The polymerase of claim 15, wherein the substitution mutation at the position functionally equivalent to Tyr409 comprises a mutation to Ala.

30. (canceled)

31. The polymerase of claim 15, wherein the substitution mutation at the position functionally equivalent to Pro410 comprises a mutation to Ile.

32. (canceled)

33. The polymerase of claim 15, wherein the substitution mutation at the position functionally equivalent to Ala485 comprises a mutation to Val.

34. (canceled)

35. The polymerase of claim 15, wherein the substitution mutation at the position functionally equivalent to Tyr497 comprises a mutation to Gly.

36. (canceled)

37. The polymerase of claim 15, wherein the substitution mutation at the position functionally equivalent to Arg247 comprises a mutation to Tyr.

38. (canceled)

39. The polymerase of claim 15, wherein the substitution mutation at the position functionally equivalent to Glu599 comprises a mutation to Asp.

40. (canceled)

41. The polymerase of claim 15, wherein the substitution mutation at the position functionally equivalent to His633 comprises a mutation to Gly.

42-90. (canceled)
